(19) United States
(12) Patent Application Publication
(10) Pub. No.: US 2024/0401152 A1
(43) Pub. Date: Dec. 5, 2024

(54) METHOD FOR DETECTING SMALL NUMBER OF FOREIGN CELLS IN CELL POPULATION WITH HIGH SENSITIVITY

(71) Applicant: ZHUHAI HENGQIN IMSTEM BIOTECHNOLOGY CO., LTD., Zhuhai, Guangdong (CN)

(72) Inventors: Xiaofang WANG, Zhuhai, Guangdong (CN); Mingsheng KONG, Zhuhai, Guangdong (CN); Hua SHEN, Zhuhai, Guangdong (CN)

(21) Appl. No.: 18/701,595

(22) PCT Filed: Oct. 18, 2022
(86) PCT No.: PCT/CN2022/125976

§ 371 Date: Apr. 15, 2024

(30) Foreign Application Priority Data
Oct. 18, 2021 (CN) 202111209270.X

(51) Int. Cl. C12Q 1/6888; C12Q 1/6851

(52) U.S. Cl. CPC C12Q 1/6888; C12Q 1/6851; C12Q 2600/158

(57) ABSTRACT

Disclosed is a method for detecting a small number of foreign cells in a cell population with high sensitivity. The method is to detect foreign cells in a sample comprising target cells and the foreign cells. In the method, one or more specific biomarkers of all cells in the sample are detected. The method comprises the step of, before extracting the total biomarkers of the sample, dividing the sample into a plurality of groups to be tested, the plurality of groups to be tested all satisfying that one foreign cell can be detected within a single system maximum sensitivity s. The detection method of the present invention can improve the sensitivity to more than one hundred thousandth.

The present application claims the priority of Chinese Patent Application No. 202111209270X filed on Oct. 18, 2021. The contents of the Chinese Patent Application are incorporated herein by reference in their entireties.

### TECHNICAL FIELD

The present disclosure belongs to the field of biochemistry, and particularly relates to a method for detecting small number of non-target cells in cell population with high sensitivity.

### BACKGROUND

With the rapid development of stem cell scientific research and cell therapy application, drug supervision departments and scientific researchers increasingly demand the quality and purity of cells. For clinical application, one of the most important problems in developing safe therapeutic cell products from pluripotent stem cells is to ensure that no tumor is formed after implantation. And one of the most significant factors is that cell products may contain contaminating undifferentiated cells, which eventually proliferate and form teratomas or other unexpected events (Carpenter, M. K., et al., Developing safe therapeutics from human pluripotent stem cells. Nature Biotechnology, 2009.27(7): p.606-613.). For life science research, ensuring that the cells do not contain non-target cells and minimizing the additional factors that interfere with the research results will greatly improve the repeatability and stability of the experiment.

Human embryonic stem cells (ESC)-mesenchymal stem cells (MSC) (hES-MSC) of ImStem Biotechnology Inc. were derived from ESC in vitro through a trophoblastic intermediate stage ([4]. Wang, X., et al., Immune modulatory mesenchymal stem cells derived from human embryonic stem cells through a trophoblast-like stage. Stem Cells, 2016. 34(2): p. 380-91.). Because pluripotent ESC can differentiate into various cell types, including tumor cells, the contamination of undifferentiated ESC in the final product of hES-MSC will cause the risk of poor cell growth or transformation. hES-MSC obtained by the method of ImStem Biotechnology Inc. is not conducive to the survival and growth of ESC due to the change of culture medium and growth environment. After multiple passages, hES-MSC is theoretically free of ESC, detection methods with high sensitivity are still required for further examination and confirmation.

At present, the conventional detection methods of cell/non-target cells are flow cytometry analysis, staining method, etc. However, because the methods are affected by many factors, such as the interference of non-specific binding of antibody dyes, and the spectral crosstalk in the flow meter, the detection sensitivity and sensitivity of these methods are limited, which cannot satisfy the needs of clinical cell therapy products or scientific research in vitro detection with high requirements for cell purity. Previously, researchers have conducted three in vitro detection methods for retinal pigment epithelial cells differentiated from human induced pluripotent stem cells (hiPS), including soft agar colony formation assay, flow cytometry and qRT-PCR. By comparison, qPT-PCR method is the most sensitive method for detection of non-target cells at present. However, their research results show that only one in ten thousandth of sensitivity can be stably achieved (Kuroda, T., et al., Highly sensitive in vitro methods for detection of residual undifferentiated cells in retinal pigment epithelial cells derived from human iPS cells. PLOS One, 2012. 7(5): p. e37342.). Typically, cell transfusion therapy requires doses of transplantation in the millions or more. It has been previously proved that hundreds of ESCs can form teratomas when transplanted subcutaneously into immunocompromised mice (Hentze, H., et al., Teratoma formation by human embryonic stem cells: Evaluation of essential parameters for future safety studies. Stem Cell Research, 2009. 2(3): p. 198-210). Although the dose of transplanted cells is adjusted to minimum, any safety risk will be reduced, the number of transplanted cells does not reach the minimum number of cells needed to produce the treatment, which will be of no benefit to patients (Hentze, H., R. Graichen and A. Colman, Cell therapy and the safety of embryonic stem cell-derived grafts. Trends in Biotechnology, 2007. 25(1): p. 24-32.). How to ensure that cell products are free of extra non-target cells, so as to ensure that cell products are as safe and effective as possible? The risk of tumorigenesis can be tested in animal models before cell transplantation. However, due to the long time of relevant animal experiments, this method may not be suitable for patients with rapidly progressive disease (Shi, Y., et al., Induced pluripotent stem cell technology: a decade of progress. Nature Reviews Drug Discovery, 2017.16(2): p. 115-130.). And the animal xenotransplantation model may not correctly reflect the long-term tumorigenic potential of human cells (Cunningham, J. J., et al., lessons from human teratomas to guide development of safe stem cell therapeutics. Nature Biotechnology, 2012.30(9): p.849-857.).

At present, the detection methods include extracting a large amount of total RNA of cells and directly diluting it for detection (or group detection). When the amount of non-target cells is very small (for example, less than 1/1,000), the detection limit of RNA is difficult to improve, and the highest detection sensitivity is only 1/100,000. Therefore, there is an urgent need for a more sensitive detection method, which can rapidly and effectively detect an extremely small number of non-target cells in the target cell population, so as to ensure that the components of cell products are as definite and reliable as possible. At present, the detection methods in the art are extracting the total RNA first and followed by group detection, but such grouping cannot improve the overall detection sensitivity.

### CONTENT OF THE PRESENT DISCLOSURE

In order to solve the defect that the lack of a high sensitivity method for detecting non- target cells in the prior art, the present disclosure provides a detection method for detecting an extremely small number of non-target cells in a cell population with high sensitivity. In the prior art, the detection of non-target cells is typically to extract specific biomarkers such as mRNA from all cells in the sample (that is, to disrupt the cells first) and then detect the biomarkers. At this moment, the content of specific biomarkers may have already been lower than the detection limit of the detection system, or the cell lysate is mixed and cannot be distinguished which cell it comes from, and each cell cannot be taken as a whole, so it is impossible to improve the overall detection sensitivity through grouping. The present invention creatively divide the cells into groups first, and then the biomarkers of cells of each group are respectively extracted for detection, and the operation of grouping first can ensure that the cells enter the reaction mix of each group to be tested as a whole, that is, each group to be tested either contains no biomarkers of non-target cells or contains at least all biomarkers of one non-target cell, so as to ensure that the content of biomarkers in each group is not lower than the detection limit of the detection system. If RNA is extracted from a large number of cells first (for example, 1 million cells) and followed by grouping the RNA, the RNA of all cells will be mixed evenly, and the RNA cannot be counted. At this moment, grouping detection (the amount of RNA corresponding to 10,000 cells/group) will be unable to prove that no non-target cell exists in each group, that is, it can only demonstrate at most that the content of non-target cells is lower than 1, rather than 0. Therefore, the detection sensitivity cannot be improved by increasing the number of groups. On the contrary, if the cells are grouped first, for example, dividing into 10,000 groups, as long as the detection by kit proves that the content of non-target cells in each group is less than one, it represents that no non-target cell exists in this group (because there is no 0.5, 0.7 or 0.9 of an entire cell). If no non-target cells existing in multiple detected groups, it represents that all the cells before grouping do not contain non-target cells (that is, the content of non-target cells is 0), so the more the groups are divided, the higher the sensitivity is. When the sensitivity of the conventional detection method is 1/10,000, the detection method of the present disclosure can improve the sensitivity to more than one in hundred thousandth.

In order to solve the above technical problems, one of the technical solutions provided by the present disclosure is: a method for detecting an extremely small number of non-target cells in a cell population with high sensitivity, which is to detect the non-target cells in a sample comprising target cells and the non-target cells, and the method detects one or more specific biomarkers of all cells in the sample; the method comprises a step of evenly dividing the sample into n groups to be tested before detecting total biomarkers of the sample, the n groups to be tested all satisfy that one of the non-target cells can be detected within a single system maximum sensitivity s, wherein n is a natural number greater than 2, preferably 10-100, such as 16.

The specific biomarkers described in the present disclosure generally refer to biological substances whose contents are quite different between target cells and non-target cells, and those skilled in the art can easily determine the specific biomarkers for detection according to the target cells to be tested and the possibly existing non-target cells. Common biomarkers comprises mRNA, DNA and protein, etc.

Preferably, the single system maximum sensitivity s is 1/10,000-1/1,000.

Preferably, the one or more specific biomarkers is mRNAs; preferably, the mRNAs comprises at least an mRNA with a large difference in expression level between the target cells and the non-target cells, and the difference in expression level is at least 100 times, such as at least 200 times, 500 times, 1,000 times, 2,000 times, 5,000 times, 7,000 times, 8,000 times, 9,000 times, 9,500 times or 10,000 times, preferably at least 1,000 times, more preferably at least 10,000 times.

In a specific example of the present disclosure, single system maximum sensitivity s is 1/1,000, and the sample has 10,000 cells in total; in the most difficult to detect situation, the sample contains one of the non-target cells, and the sample can be divided into 10 groups to be tested, and the total biomarkers in each group to be tested are detected, only one group to be tested contains the non-target cell, and this group can detect the specific biomarkers of the amount of one non-target cell under the single system maximum sensitivity s, while the remaining groups to be tested do not contain the specific biomarkers. Combining the results of the 10 groups to be tested can improve the total system maximum sensitivity to 1/10,000 under the situation that the single system maximum sensitivity s is 1/1,000 and the sample has 10,000 cells in total.

In a specific example of the present disclosure, the single system maximum sensitivity s is 1/10,000, the sample has 10,000,000 cells in total; in the most difficult to detect situation, the sample contains one of the non-target cells, and the sample can be divided into 1,000 groups to be tested, and the total biomarkers in each group to be tested are detected, only one group to be tested contains the non-target cell, and this group can detect the specific biomarkers of the amount of one non-target cell under the single system maximum sensitivity s, while the remaining groups to be tested do not contain the specific biomarkers. Combining the results of 1,000 groups to be tested can improve the total system maximum sensitivity to 1/10,000,000 under the situation that the single system maximum sensitivity s is 1/10,000 and the sample has 10,000,000 cells in total.

Preferably, the single system maximum sensitivity s is determined by an expression level of the one or more specific mRNAs, a sensitivity of a detection instrument and a primer and/or a probe designed for one or more specific mRNAs. Those skilled in the art can determine the single system maximum sensitivity s by literature search, pilot experiment and other methods according to the actual situation.

Preferably, the detection instrument is a real-time fluorescence quantitative PCR instrument, such as StepOnePlus of Thermo Fisher Scientific; and/or the method is conducted with a single-cell mRNA amplification kit.

Preferably, the primer has cross-intron design; Tm of the probe is 68-72° C. Preferably, the probe is a Taqman probe, the cross-intron design is beneficial to more accurate collocative detection of the primer and the Taqman probe.

In a specific example of the present disclosure, the non-target cell is human embryonic stem cell line 053 and the target cell is human umbilical cord mesenchymal stem cell TMSC.

Preferably, the mRNA with a large difference in the expression level is an mRNA transcribed from OCT4 gene.

In a specific example of the present disclosure, the sequences of the primer are shown in SEQ ID NO: 1 and SEQ ID NO: 2, the probe is a Taqman probe, the sequence of the probe is shown in SEQ ID NO: 3.

In a specific example of the present disclosure, the method further comprises a positive control group set according to the single system maximum sensitivity, the positive control group comprises at least one specific biomarker, such as mRNA, of an amount of one non-target cell, and a total number of cells in the positive control group is the same as a number of cells of a single one of the groups to be tested.

On the basis of common sense in the field, the above-mentioned preferred conditions can be arbitrarily combined to obtain the preferred examples of the present disclosure.

The reagents and raw materials used in the present disclosure are available in the market.

The present disclosure has the following positive and progressive effects:

The present disclosure provides a method for detecting an extremely small number of non-target cells in a cell population with high sensitivity, the sensitivity of the method is higher than the known sensitivity of one in ten thousandth for non-target cells detection, and preferably it can reach one in millionth or more. The detection cost of the method is lower, and the operation is simpler and faster. When the method detects the tumorigenicity of sample cells to be tested, the results can reflect the tumorigenicity and safety of cell products to a certain extent. In addition, the method can also be used to detect a small number of other cell population accidentally introduced into the target cell population, which helps to understand the homogeneity of the target cell population.

### BRIEF DESCRIPTION OF THE DRAWINGS

FIG. 1A is a curved graph of qPCR fluorescence amplification signal of ACTB in each experimental group. The initial template amount of each group is cDNA of 10,000 cells. The sample groups represented by each curve have been indicated by arrows or braces in the figure.

FIG. 1B is a curved graph of qPCR fluorescence amplification signal of OCT4 in each experimental group. The initial template amount of each group is cDNA of 10,000 cells. The sample groups represented by each curve have been indicated by arrows or braces in the figure.

FIG. 2A is a curved graph of qPCR fluorescence amplification signal of ACTB in each experimental group. The initial template amount of each group is cDNA of 10,000 cells. The sample groups represented by each curve have been indicated by arrows or braces in the figure.

FIG. 2B is a curved graph of qPCR fluorescence amplification signal of OCT4 in each experimental group. The initial template amount of each group is cDNA of 10,000 cells. The sample groups represented by each curve have been indicated by arrows or braces in the figure.

FIG. 3A is a curved graph of qPCR fluorescence amplification signal of ACTB in each experimental group. The initial template amount of each group is cDNA of 10,000 cells. The sample groups represented by each curve have been indicated by arrows or braces in the figure.

FIG. 3B is a curved graph of qPCR fluorescence amplification signal of OCT4 in each experimental group. The initial template amount of each group is cDNA of 10,000 cells. The sample groups represented by each curve have been indicated by arrows or braces in the figure.

### DETAILED DESCRIPTION OF THE EMBODIMENT

The present disclosure is further illustrated below by ways of the examples, but the present disclosure is not thereby limited to the scope of the described examples. The experimental methods of which the specific conditions are not specified in the following examples shall be selected according to the conventional methods and conditions, or according to the product instruction.

### Experimental Supplies

(1) Experimental instruments and equipment are shown in Table 1 below:

TABLE 1
Instruments and Equipment
Name of instruments and equipment

Real-time fluorescence quantitative PCR instrument
(Thermo Fisher Scientific, StepOnePlus)

(2) Experimental reagents and cell samples are shown in Table 2 below:

TABLE 2
Experimental Reagents and Cell Samples

| Name of reagents | Source (brand and catalog no./batch no.) |
|---|---|
| Single Cell Sequence Specific | Vazyme, P621-01 |
| Amplification Kit | |
| AceQ^{R} Universal U + | Vazyme, Q513-02 |
| Probe Master Mix V2 | |
| DPBS | GIBCO, 2235070 |
| Human embryonic stem cell | ESI BIO, ES-704 |
| line (053-ESC, hereafter | |
| referred to as 053) | |
| Human umbilical cord | Prepared in laboratory (Reference: Duo Li. Study |
| mesenchymal stem | on isolation and culture method of human |
| cell (UCMSC) | umbilical cord mesenchymal stem cell [D]. |
| | Peking Union Medical College, 2011.) |
| Cells to be tested (hES-MSC, | Prepared in laboratory (Reference: Wang, X., et |
| hereafter referred to as TMSC) | al., Immune modulatory mesenchymal stem cells |
| | derived from human embryonic stem cells through |
| | a trophoblast-like stage. Stem Cells, 2016. 34(2): |
| | p. 380-91.) |
| Trypan blue staining | GIBCO, 15250-061 |

(3) Sequence information of RT-qPCR primers and probes are shown in Table 3 below:

TABLE 3
Sequence information of RT-qPCR primers and probes

| Name of primer and probe | Sequence | SEQ ID NO: |
|---|---|---|
| OCT4A forward primer | GCTTGGAGACCTCTCAGCCT | 1 |
| OCT4A reverse primer | TTGATGTCCTGGGACTCCTC | 2 |
| OCT4A probe | FAM-CAGGGGTGACGGTG-BHQ1 | 3 |
| ACTB forward primer | GCACAGAGCCTCGCCTTTG | 4 |
| ACTB reverse primer | ATCCATGGTGAGCTGGCG | 5 |
| ACTB probe | FAM-TGTGGACGGGCGGCGGATC-BHQ1 | 16 |

Determination of mRNA with a large difference in expression level between 053-ESC and UCMSC: OCT4A is a key component of pluripotent ES cell regulatory network. OCT4A gene is a member of POU transcription factor family, which is mainly expressed in embryonic stem cells, germline stem cells and undifferentiated embryonic carcinoma. RT-PCR can be used for quantitative and qualitative analysis of the transcription amount of OCT4A in cells ([5]. Ren, J.-j. and X.-k. Meng, A relative quantitative method to detect OCT4A gene expression by exon-junction primer and locked nucleic acid-modified probe. Journal of Zhejiang University. Science. B, 2011. 12(2): p. 149-155.). However, the expression level of OCT4A in adult MSC is relatively low, so the content ratio of OCT4A can be obtained by RT-PCR to identify the proportion of ES cells in MSC cells and to confirm whether the purity of cultured MSC cells satisfies the requirements. For example, from the known 10,000 cells, the relevant content cannot be detected by the reaction mix of the present disclosure, indicating that its expression content has been lower than the detection limit. When the reaction mix of the present disclosure is used to detect the positive control, the highest content can be detected is 1/40,000. This indicates that the expression level of OCT4 gene in UCMSC is at least 40,000 times lower than that in 053ESC.

Calculation and analysis of single system maximum sensitivity

According to the preliminary experiment, under the current system, when the mRNA content of OCT4A is 1/10,000 (that is, a sample of 10,000 cells contains one 053-ESC), a stable signal can be detected, so the single maximum sensitivity of the experimental system is determined to be one in ten thousandth.

### Example 1 The number of TMSC Groups (N) to be Tested is 3

### I. Preparation of Cell Samples

Conventional materials (1.5 ml EP tubes, pipettes, etc.) needed for cell processing were prepared 30-60 minutes in advance and put in the biological safety cabinet, and the ultraviolet lamp was turn on for sterilization. PBS was taken out from the refrigerator and incubated at room temperature for 10-30 min. The water bath was turned on and the water temperature was adjusted to 37° C. After the ultraviolet irradiation was done, three clean EP tubes were taken out and labeled 053, UCMSC and TMSC, respectively.

Three kinds of cells, 053, UCMSC and TMSC, were taken out from liquid nitrogen or −80° C. ultra-low temperature freezer. The cells were transferred to a water bath for quick thawing. The cells were then transferred to the clean bench, 3-6 ml PBS was added into each group and mixed evenly, and the mixture was centrifuged at 200 g for 5 minutes. The supernatant was carefully absorbed with a pipette. Three new EP tubes were taken out, 10 μL PBS was added to each tube, and the tubes were labeled 053 (count), UCMSC (count) and TMSC (count).

In each group, an appropriate amount of PBS was added to resuspend the cells (the cell density was more than 2.5×106/ml by estimation), and 5 μL cell suspension was absorbed into 10 μL PBS as soon as possible to mix well, and then 10 μL trypan blue was added to mix well. At this point, the cell density had been diluted by 5 times. The cell count was carried out by a hemocytometer, and the density of the cell stock solution was obtained by multiplying the counting result by the dilution multiple of 5. The cell viability was ensured to be above 90%, otherwise the cells would be revived again according to the above steps.

According to the cell density of the stock solution, an appropriate volume of PBS was added to adjust the cell density of each group to 2.5×106/ml for later use.

Because 053 cells are prone to apoptosis, the detection effect will thereby be affected, resulting in false negative. Therefore, it is necessary to treat cells as soon as possible to ensure that the viability of cell samples is above 90% and proceed to the next step as soon as possible.

### II. Preparation of Cell Sample cDNA of Each Group

1. Preparation of 0.1 μM Reaction Mix is Shown in Table 4 Below:

TABLE 4
Preparation of reaction mix

| | |
|---|---|
| OCT4A forward primer (50 μM) | 2 μL |
| OCT4A reverse primer (50 μM) | 2 μL |
| ACTB forward primer (50 μM) | 2 μL |
| ACTB reverse primer (50 μM) | 2 μL |
| ddH₂O | adding up |
| | to 1,000 μL |

After preparation, it can be separated and frozen in the −20° C. freezer for multiple uses.

The pre-mixed solution (the above is the added volume of each component in a single well, and the added volume of the reagent can be proportionally expanded according to the specific number of lysis wells) of system (cell-free) is prepared and then the pre-mixed liquid were separated into 21 μl to prepare wells. 4 cell samples were then added to each hole. Before adding, the cell suspension was blown evenly to reduce the sampling error. The addition of cells into the system needs to be fast, and the addition of all cell samples was finished within 5 min and the temperature was kept low to minimize the degradation of RNA groups in cells. After the cells were added and mixed evenly, the container was immediately transferred to the −80° C. freezer for freezing for 2 min. After taking out the sample, the sample was centrifuged at 3,000 rpm for 2 min and RT-PCR reaction (reverse transcription polymerase chain reaction) was immediately carried out.

2. RT-PCR reaction mix is shown in Table 5 below:

TABLE 5
RT-PCR reaction mix

| | | |
|---|---|---|
| 2 × Reaction Mix | 12.5 | μL |
| 0.1 μM Assay Pool | 2.5 | μL |
| RT/Taq polymerase | 0.5 | μL |
| cell | 4 | μL |

| | |
|---|---|
| ddH₂O | adding up |
| | to 25 μL |

RT-PCR reaction process:

Process is set as below:

| | | | | |
|---|---|---|---|---|
| 50° C. | 60 | min | | |
| 95° C. | 3 | min | | |
| 95° C. | 15 | s | | 17 cycles |
| | | | {close oversize brace} | |
| 60° C. | 8 | min | | |

| | |
|---|---|
| 4° C. | hold |

After the reaction, the cDNA sample was taken out as soon as possible, and the sample was frozen and stored in the −80° C. freezer for later use, or the next-step qPCR experiment was immediately carried out.

3. Thawing and addition of cDNA sample:

Each group of cDNA (25 μL/group) was thawed on ice.

Illustration of the ratio of positive dilution control group:

The two positive groups (053-ESC) were first mixed evenly to reduce the fluctuation and error of the positive group during the preparation. Then the mixed pure positive group was diluted with each UCMSC group according to the following sampling volume.
25×(1 μL 053-ESC cDNA+24 μL UCMSC cDNA), i.e. 400ESC+9600 UCMSC
625×(1 μL 25×cDNA+24 μL UCMSC cDNA), i.e. 16ESC+9984 UCMSC
5,000×(3 μL 625×cDNA+21 μL UCMSC cDNA), i.e. 2ESC+9998 UCMSC (this group is an optional group)
10,000×(1.5 μL 625×cDNA+22.5 μL UCMSC cDNA), i.e. 1ESC+9999 UCMSC
Negative control group: H₂O, UCMSC (cDNA).
Sample to be tested groups: TMSC1 (cDNA), TMSC2 (cDNA), TMSC3 (cDNA).
Pure positive group: 053(cDNA).

The specific grouping was as follows:

Thawing and addition of cDNA sample:

The experimental grouping are shown in Table 6 and Table 7 below:

TABLE 6
positive control group

| Gene | Positive control group | | | | |
|---|---|---|---|---|---|
| OCT4 | 053-ESC | UCMSC:ESC = | UCMSC:ESC = | UCMSC:ESC = | UCMSC:ESC = |
| | | 10,000:400 | 10,000:16 | 10,000:2 | 10,000:1 |
| | | (25×) | (625×) | (5,000×) | (10,000×) |
| | 053-ESC | UCMSC:ESC = | UCMSC:ESC = | UCMSC:ESC = | UCMSC:ESC = |
| | | 10,000:400 | 10,000:16 | 10,000:2 | 10,000:1 |
| | | (25×) | (625×) | (5,000×) | (10,000×) |
| ACTB | 053-ESC | UCMSC:ESC = | UCMSC:ESC = | UCMSC:ESC = | UCMSC:ESC = |
| | | 10,000:400 | 10,000:16 | 10,000:2 | 10,000:1 |
| | | (25×) | (625×) | (5,000×) | (10,000×) |
| | 053-ESC | UCMSC:ESC = | UCMSC:ESC = | UCMSC:ESC = | UCMSC:ESC = |
| | | 10,000:400 | 10,000:16 | 10,000:2 | 10,000:1 |
| | | (25×) | (625×) | (5,000×) | (10,000×) |

TABLE 7
to be tested group and negative control group

| Gene | To be tested group × N | | | Negative control group | |
|---|---|---|---|---|---|
| OCT4 | TMSC-1 | TMSC-2 | TMSC-3 | UCMSC | H₂O |
| | TMSC-1 | TMSC-2 | TMSC-3 | UCMSC | H₂O |
| ACTB | TMSC-1 | TMSC-2 | TMSC-3 | UCMSC | H₂O |
| | TMSC-1 | TMSC-2 | TMSC-3 | UCMSC | H₂O |

N in Table 7 is a natural number, and different groups of TMSC can be detected according to the specific experimental purpose. The detection sensitivity is 10,000×N.

5 μL cDNA was taken from all the above-mentioned groups, with addition of 70 μL ddH2O respectively. At this moment, cDNA of each group had been diluted by 15 times, and 5 μL diluted cDNA was added to each reaction mix and mixed evenly.

The remaining cDNA groups can be separated and frozen in the −80° C. freezer for later use or retest.

Each positive control group (cDNA) and negative group UCMSC (cDNA) can be separated and frozen in a −80° C. freezer for multiple use under the situation that the operation and the kit used in each experiment are basically consistent. Under the situation that positive control groups and negative groups are sufficient, only the cDNA of the group to be tested needs to be prepared for each detection of TMSC.

### III. qPCR Detection

qPCR detection:

Preparation of Primer Mi × (10 μM) + Probe (5 μM)

| | |
|---|---|
| forward primer (50 μM) | 100 μL |
| reverse primer (50 μM) | 100 μL |
| Probe (25 μM) | 100 μL |
| ddH₂O | 200 μL |

After mixed evenly, the qPCR reaction mix was separated into 20 μL/tube, and was stored frozen in a −20° C. freezer away from light, and thawed on ice before use.

qPCR reaction mix (away from light), shown in Table 8 below:

TABLE 8
qPCR reaction mix

| | | |
|---|---|---|
| 2 × Probe Master Mix V2 | 10 | μL |
| Primer Mix (10 μM) + | 0.5 | μL |
| Probe (5 μM) | | |
| cDNA | 5 | μL |

| | |
|---|---|
| ddH₂O | Up to 20 μL |

OCT4 detection pre-mixed solution (not containing cDNA) group and ACTB pre-mixed solution group were prepared respectively, 15 μL pre-mixed solution was added into each tube and 5 μL diluted cDNA sample was finally supplemented to each tube.

qPCR reaction process:

Process is set as below:

| | | | | |
|---|---|---|---|---|
| 37° C. | 2 | min | | |
| 95° C. | 5 | min | | |
| 95° C. | 10 | s | | 45 cycles |
| | | | {close oversize brace} | |
| 60° C. | 30 | s | | |

Fluorescent signal is collected at 60° C. 30 s.

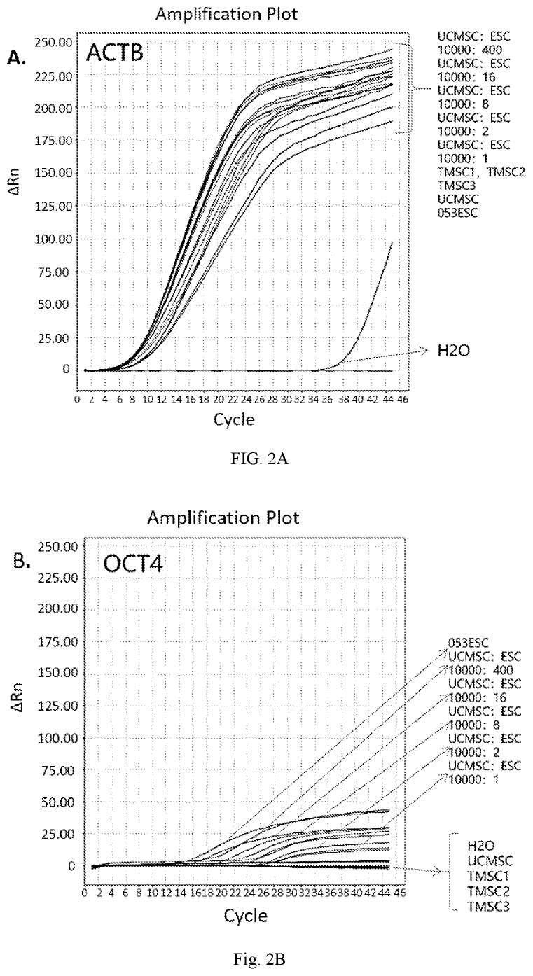

When the number of TMSC groups to be tested (N) is 3, qPCR fluorescence amplification signal curve was as shown in FIG. 2.

Three groups to be tested were designed in Example 1, namely TMSC1, TMSC2 and TMSC3, respectively. The group to be detected derived from the same batch of TMSC cells, and about 10,000 TMSC cells were taken from each group to be detected. According to the experimental steps, the cell grouping would be affected by the accuracy of pipette, random error of cells, etc. The number of cells might have an error of ±5%. It was generally believed that the error of ±5% of the total number of cells would not generate a significant impact on the signal value of 1 non-target cell.

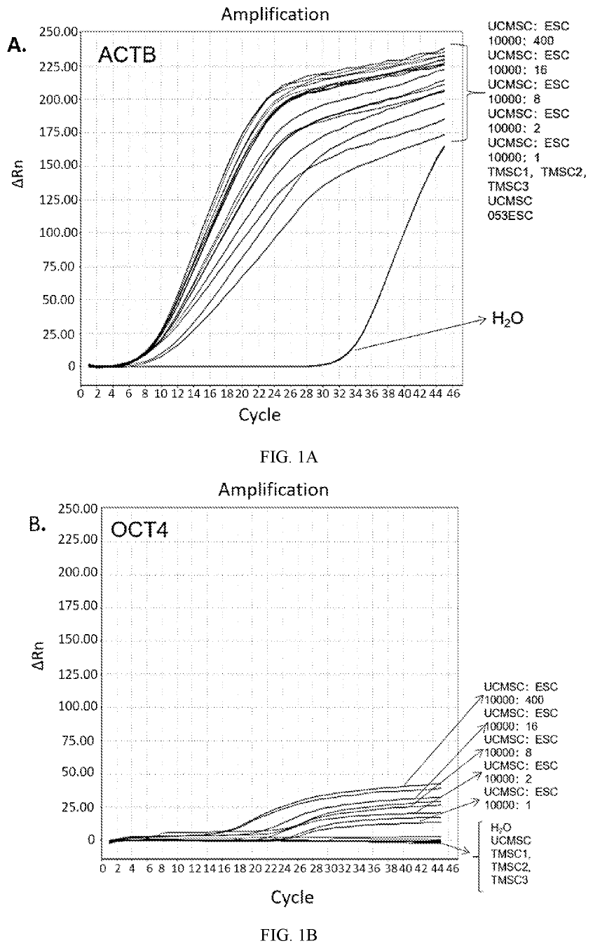

As shown in FIG. 1A, the CT value of ACTB in negative control group H₂O was 30, and while the CT values of ACTB in other groups added with cell cDNA were all in the range of 2-8, indicating that the differences in the amount of cDNA templates between cell samples of each group are small. Due to the small differences between groups, the groups and curves are not distinguished one by one in the figure. As shown in FIG. 1B, OCT4 was detected in all negative control group and each group to be tested, while the CT value of maximum dilution ratio UC-MSC: ESC (10,000:1) group in positive control group was 24. Therefore, the OCT4 content of negative control group (UC-MSC) and group to be tested (hES-MSC) was less than that of the maximum dilution ratio UC-MSC: ESC (10,000:1) group in positive control group. The results show that the content of ESC cells in the three groups to be tested was lower than 1 ESC, that is, the detected 10,000×3 hES-MSC cells do not contain one ESC non-target cell. Therefore, it can be obtained that the detection sensitivity is one of thirty thousand.

### Example 2 Number of TMSC Groups (N) to be Tested is 3

The experimental method was the same as that in Example 1. Three groups to be tested were designed in Example 2, namely TMSC1, TMSC2 and TMSC3, respectively. The group to be tested derived from the same batch of TMSC cells, and about 10,000 TMSC cells were taken from each group to be tested.

As shown in FIG. 2A, the CT value of ACTB in negative control group H₂O is 34, while the CT values of AC TB in other groups added with cell cDNA are all in the range of 2-6, indicating that the differences in the amount of cDNA templates between cell samples in each group are small. Due to the small differences between groups, the groups and curves are not distinguished one by one in the figure. As shown in FIG. 2B, OCT4 was detected in all the negative control group and each group to be tested, while the CT value of maximum dilution ratio UC-MSC: ESC (10,000:1) group in positive control group is 26. Therefore, the OCT4 content of negative control group (UC-MSC) and group to be tested (hES-MSC) is less than that of maximum dilution ratio UC-MSC: ESC (10,000:1) group in positive control group. The results show that the content of ESC cells in the three groups to be tested was lower than 1 ESC, that is, the detected 10,000×3 hES-MSC cells do not contain one ESC non-target cell. Therefore, it can be obtained that the detection sensitivity is 1/30,000.

The result of the present experiment is similar to that of Example 1. It can be known that the present detection method is less affected by system errors and could maintain high stability in detection with high sensitivity.

### Example 3 Number of the TMSC Groups (N) to be Tested is 16

The experimental method was the same as that in Example 1. 16 groups to be tested were designed in Example 3, namely TMSC1, TMSC2, TMSC3 . . . TMSC16, respectively. Different from Example 1 and Example 2, the cDNA of the present positive control group and the cDNA of the negative control group UCMSC were prepared in Example 2. Previously, the CNDA template was frozen in a −80° C. freezer and thawed again when used, while the cDNA of TMSC cells in the group to be tested was freshly prepared. The group to be tested derived from the same batch of TMSC cells, and about 10,000 TMSC cells were taken from each group to be tested.

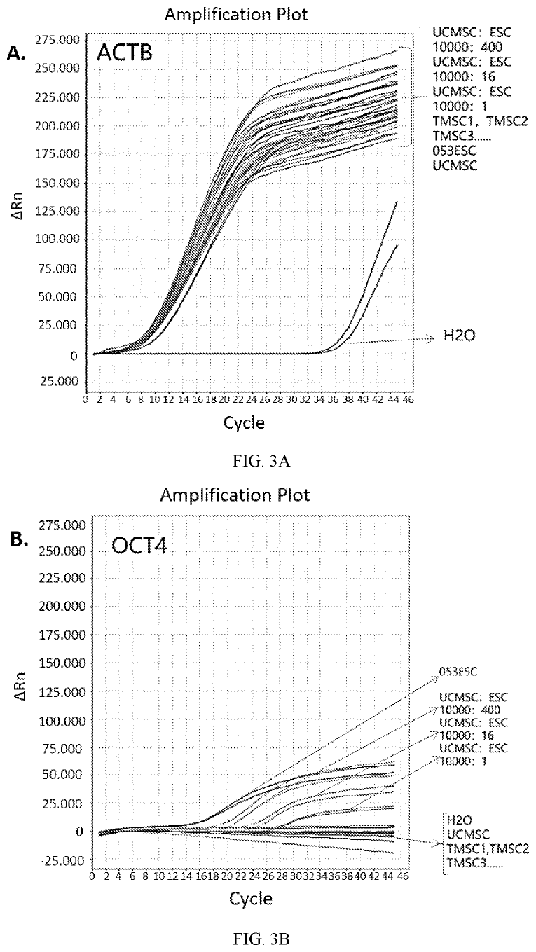

As shown in FIG. 3A, the CT value of ACTB in negative control group H₂O is 34, while the CT values of ACTB in other groups added with cell cDNA are all in the range of 2-6, indicating that the differences in the amount of cDNA templates between cell samples in each group are small. Due to the small differences between groups, the groups and curves are not distinguished one by one in the figure. As shown in FIG. 3B, OCT4 was detected in all negative control group and each group to be tested, while the CT value of maximum dilution ratio UC-MSC: ESC (10,000:1) group in positive control group is 28. Therefore, the OCT4 content of negative control group (UC-MSC) and group to be tested (hES-MSC) is less than that of the maximum dilution ratio UC-MSC: ESC (10,000:1) group in positive control group. The results show that the content of ESC cells in 16 groups to be tested is lower than 1 ESC, that is, the detected 10,000×16 hES-MSC cells do not contain one ESC non-target cell. Therefore, it can be obtained that the detection sensitivity is 1/160,000.

In addition, according to the comparison between the curve results of ACTB and OCT4 of cDNA of positive control group and negative control group in Example 2 and Example 3, it can be found that the difference is small. That is, the stability of samples' cDNA of each group would not be obviously affected after freezing and thawing and storage in a −80° C. freezer for at least one week. Therefore, the cDNA of positive group and negative group could be prepared in advance and stored in the −80° C. ultra-low temperature freezer for the use of subsequent detection.

From the above examples, it can be known to those skilled in the art that when the number of groups to be tested is N, the detection sensitivity is 1/10,000 times N, and the number of cell groups to be tested can be appropriately increased or decreased according to the purpose of the experiment.

Finally, the experimenter can increase the number of groups to be tested according to the sensitivity requirements and operating proficiency. When samples are added manually, it is necessary to be as accurate as possible and to operate as fast as possible to avoid the operation error between groups. If conditions permit, it is necessary to increase the number of groups (N is greater than 17) to reach a higher detection level. It is suggested to use automatic and batch sampling instruments and equipment to add reagents, in order to achieve a smaller error between groups, greatly increase the number of groups to be tested, improve the detection sensitivity, and ensure the accuracy of the results.

1. A method for detecting an extremely small number of non-target cells in a cell population with high sensitivity, the method is to detect the non-target cells in a sample comprising target cells and the non-target cells, wherein the method detects one or more specific biomarkers of all cells in the sample; the method comprises a step of evenly dividing the sample into n groups to be tested before detecting total biomarkers of the sample, the n groups to be tested all satisfy that one of the non-target cells can be detected within a single system maximum sensitivity s, wherein n is a natural number greater than 2.

2. The method of claim 1, wherein the single system maximum sensitivity s is 1/100,000-1/100.

3. The method of claim 1, wherein the one or more specific biomarkers is mRNAs.

4. The method of claim 1, wherein the single system maximum sensitivity s is determined by an expression level of the one or more specific mRNAs, a sensitivity of a detection instrument, and a primer or a probe designed for one or more specific mRNAs.

5. The method of claim 4, wherein the detection instrument is a real-time fluorescence quantitative PCR instrument, or the method is conducted with a single-cell mRNA amplification kit.

6. The method of claim 4, wherein the primer has a cross-intron design; Tm of the probe is 68-72° C.

7. The method of claim 3, wherein the non-target cells are human embryonic stem cell line 053, the target cells are human umbilical cord mesenchymal stem cells TMSC.

8. The method of claim 3, wherein the mRNA with a large difference in expression level is an mRNA transcribed from OCT4 gene.

9. The method of claim 8, wherein the sequences of the primer are shown in SEQ ID NO: 1 and SEQ ID NO: 2, the probe is a Taqman probe, the sequence of the probe is shown in SEQ ID NO: 3.

10. The method of claim 1, wherein the method further comprises a positive control group set according to the single system maximum sensitivity, the positive control group comprises at least one specific biomarker, of an amount of one non-target cell, and a total number of cells of the positive control group is the same as a number of cells of a single one of the groups to be tested.

11. The method of claim 10, wherein the specific biomarker is mRNA.

12. The method of claim 5, wherein the real-time fluorescence quantitative PCR instrument is StepOnePlus of Thermo Fisher Scientific Inc.

13. The method of claim 3, wherein the mRNAs comprise at least an mRNA with a large difference in expression level between the target cells and the non-target cells, and the difference in expression level is at least 100 times.

14. The method of claim 13, wherein the difference in expression level is at least 1,000 times.

15. The method of claim 14, wherein the difference in expression level is at least 10,000 times.

16. The method of claim 2, wherein the single system maximum sensitivity s is 1/10,000-1/1,000.

17. The method of claim 1, wherein n is 10-1000.

18. The method of claim 17, wherein n is 16.


SEQUENCE LISTING (6 sequences)

SEQ ID NO: 1 - DNA, 20 bp, synthetic construct
  misc_feature 1..20: note = OCT4A
  source 1..20: mol_type = other DNA
gcttggagacctctcagcct

SEQ ID NO: 2 - DNA, 20 bp, synthetic construct
  misc_feature 1..20: note = OCT4A
  source 1..20: mol_type = other DNA
ttgatgtcctgggactcctc

SEQ ID NO: 3 - DNA, 14 bp, synthetic construct
  misc_feature 1..14: note = OCT4A
  misc_binding 1: bound_moiety = FAM
  misc_binding 14: bound_moiety = BHQ1
  source 1..14: mol_type = other DNA
caggggtgacggtg

SEQ ID NO: 4 - DNA, 19 bp, synthetic construct
  misc_feature 1..19: note = ACTB
  source 1..19: mol_type = other DNA
gcacagagcctcgcctttg

SEQ ID NO: 5 - DNA, 18 bp, synthetic construct
  misc_feature 1..18: note = ACTB
  source 1..18: mol_type = other DNA
atccatggtgagctggcg

SEQ ID NO: 6 - DNA, 19 bp, synthetic construct
  misc_feature 1..19: note = ACTB
  misc_binding 1: bound_moiety = FAM
  misc_binding 14: bound_moiety = BHQ1
  source 1..19: mol_type = other DNA
tgtggacgggcggcggatc